(19) United States
(12) Patent Application Publication
(10) Pub. No.: US 2016/0214941 A1
(43) Pub. Date: Jul. 28, 2016

(54) Compounds for lnhibition of Unregulated Cell Growth

(71) Applicant: GODAVARI BIOREFINERIES LIMITED, Maharashtra (IN)

(72) Inventors: Kailas Pangavhane, Mumbai (IN); Maithill Athavale, Mumbai (IN)

(21) Appl. No.: 15/024,980

(22) PCT Filed: Sep. 26, 2014
(86) PCT No.: PCT/IN14/00622

§ 371 Date: Mar. 25, 2016

(30) Foreign Application Priority Data
Sep. 26, 2013 (IN) 3015/MUM/2013

(51) Int. Cl. C07D 219/10; A61K 45/06; A61K 31/473

(52) U.S. Cl. CPC C07D 219/10; A61K 31/473; A61K 45/06

(57) ABSTRACT

Compounds For Inhibition Of Unregulated Cell Growth The present invention relates to compounds of Formula I for inhibition or eradication of unregulated cell growth.

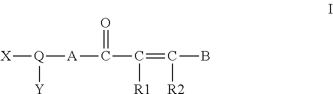

### FIELD OF THE INVENTION

The invention relates to compounds for the inhibition or eradication of unregulated cell, growth.

### BACKGROUND OF THE INVENTION

Cancer is a condition in which abnormal cells proliferate and spread anywhere in the body. In other words, cancer is an uncontrolled growth of abnormal cells. Cancer is a leading cause of death worldwide. It is of major concern in India and is reported to be one of the ten leading causes of deaths in India. As per WHO Report 2005, the cancer deaths in India are estimated to increase to 7 lakh by 2015. The World Health Organization lists the following facts about cancer:

Cancer is a leading cause of death worldwide, accounting for 7.6 million deaths (around 13% of all deaths) in 2008.

The main types of cancer are:
lung (1.37 million deaths)
stomach (736 000 deaths)
liver (695 000 deaths)
colorectal (608 000 deaths)
breast (458 000 deaths)
cervical cancer (275 000 deaths)

There are various types of cancers, which can fit in the below general categories as per National Cancer Institute.
Carcinoma: Cancer that begins in the skin or in tissues that line or cover internal organs
Sarcoma: Cancer that begins in bone, cartilage, fat, muscle, blood vessels, or other connective or supportive tissue
Leukemia: Cancer that starts in blood-forming tissue such as the bone marrow and causes large numbers of abnormal blood cells to be produced and enter the blood
Lymphoma and myeloma: Cancers that begin in the cells of the immune system
Central nervous system cancers: Cancers that begin in the tissues of the brain and spinal cord

The leading causes of cancer are the use of tobacco, alcohol, unhealthy diet, chronic infections, physical inactivity, etc.

The present course of treatment for cancer usually depends on the type and stage of cancer. The most common type of treatment is surgery, radiotherapy, chemotherapy or a combination of these therapies. Palliative treatments are also available to reduce cancer symptoms.

Though medications are available to treat cancer drugs, common side effects of these readily available drugs include nausea and vomiting, loss of appetite, change in taste, thinned or brittle hair, pain in the joints of the aims or legs, changes in the color of the nails, and tingling in the hands or toes. More serious side effects such as unusual bruising or bleeding, pain/redness/swelling at the injection site, change in normal bowel habits for more than two days, fever, chills, cough, sore throat, difficulty swallowing, dizziness, shortness of breath, severe exhaustion, skin rash, facial flushing, female infertility by ovarian damage and chest pain can also occur. (Ozcelik, Bulent; Turkyilmaz, Cagdas; Ozgun, Mahmut Tuncay; Serin, Ibrahim Serdar; Batukan, Cem; Ozdamar, Saim; Ozturk, Ahmet (2010). “Prevention of paclitaxel and cisplatin induced ovarian damage in rats by a gonadotropin-releasing hormone agonist”. Fertility and Sterility 93 (5): 1609-14)

Inorder to mitigate the side effects of commercially available drugs, extensive research is being done by altering the administration; these drugs target only cancer cells and not cancer stem cells. Thus there is a high probability of relapse of the cancer. Nonetheless, there is a need to develop drugs which can inhibit, control and eliminate the cancer cells along with cancer stem cells with minimum side effects.

Cancer stem cells (CSCs) are cancer cells (found within tumors or hematological cancers) that possess the capacity to self-renew and to cause the heterogeneous lineages of cancer cells that comprise the tumor (Clarke M F, et al. Cancer Stem Cells—Perspectives on Current Status and Future Directions: AACR Workshop on Cancer Stem Cells. Cancer Res. 2006; 66:9339-9344). CSCs are therefore tumorigenic (tumor-forming), in contrast to other non-pluripotent cancer cells. CSCs may generate tumors through the stem cell processes of self-renewal and differentiation into multiple cell types. Such cells are proposed to persist in tumors as a distinct population and cause relapse and metastasis by giving rise to new tumors. Therefore, development of specific therapies targeted at CSCs holds hope for improvement of survival and quality of life of cancer patients, especially for sufferers of drug-resistant tumors or metastatic disease.

The continuing problems of drug resistance and tumor relapse following traditional first-line cancer chemotherapy, and the current lack of drugs which effectively kill CSCs highlight the overwhelming need for new inhibitors of CSC proliferation.

### SUMMARY OF THE INVENTION

An aspect of the present invention relates to compounds of Formula I for treating various conditions, particularly for inhibiting unregulated cell growth is provided. The structure of Formula I is as follows:

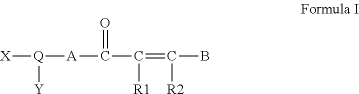

wherein,

X is substituted heterocyclic ring, substituted tricyclic ring with a heteroatom, preferably N

Q is preferably N

Y is H

A is substituted or unsubstituted aromatic ring, substituted or unsubstituted aromatic ring with a heteroatom

R1 and R2, each independently is H

B is a substituted or unsubstituted aromatic ring.

Another aspect of the present invention discloses a process for preparing a compound for inhibition of unregulated cell growth. The process comprises of reacting compound of Formula V with a heterocyclic compound having N as a heteroatom to form a mixture and
refluxing the mixture in the presence of a solvent to obtain the compound.

### BRIEF DESCRIPTION OF THE DRAWINGS

FIG. 1 and FIG. 2 illustrate unstained PC3 population.

FIG. 3 & FIG. 4 illustrate PC3 cells with CD24 and CD44 antibodies.

FIG. 5 and FIG. 6 illustrate PC3 cells exposed to IC 10 Cisplatin.

FIG. 7 and FIG. 8 illustrate PC3 cells exposed to IC25 Cisplatin.

FIG. 9 and FIG. 10 illustrate PC3 cells exposed to IC10 of compound of Formula IV.

FIG. 11 and FIG. 12 illustrate PC3 cells exposed to IC25 of compound of Formula IV.

FIG. 13 illustrates effect of Cisplatin and compounds of Formula III and IV on sphere formation of MDA MB 231 cell line.

### DESCRIPTION OF THE INVENTION

According to various embodiments of the present invention that are described below, compounds of Formula I for treating various conditions, particularly for inhibiting unregulated cell growth is provided. The structure of Formula I is as follows:

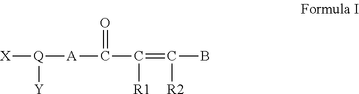

wherein,

X is substituted or unsubstituted heterocyclic ring, substituted or unsubstituted tricyclic ring with a heteroatom, preferably N,

Q is preferably N,

Y is H,

A is substituted or unsubstituted aromatic ring, substituted or unsubstituted aromatic ring with a heteroatom,

R1 and R2, each independently is H, and

B is a substituted or unsubstituted aromatic ring

Particularly, the present invention relates to compounds of Formula II for treating various conditions, particularly for inhibition or eradication of unregulated cell growth, represented by the following structure:

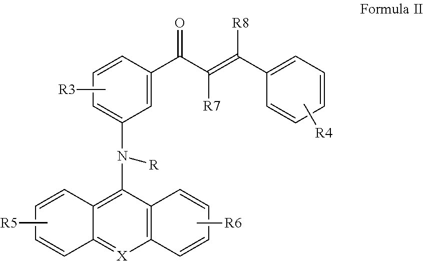

wherein,

R3 is H, I, Br, alkoxy group, optionally substituted alkyl group,

R4, R5 and R6, each independently is alkoxy group, I, Cl, Br, CN, NO2, optionally substituted alkyl group,

R7 and R8 each independently is H,

X is N, and

R is H, HCl, H2SO4 or salts thereof.

Preferably, the compounds for treating various conditions, particularly for inhibition or eradication of unregulated cell growth are represented by Formula III and Formula IV:

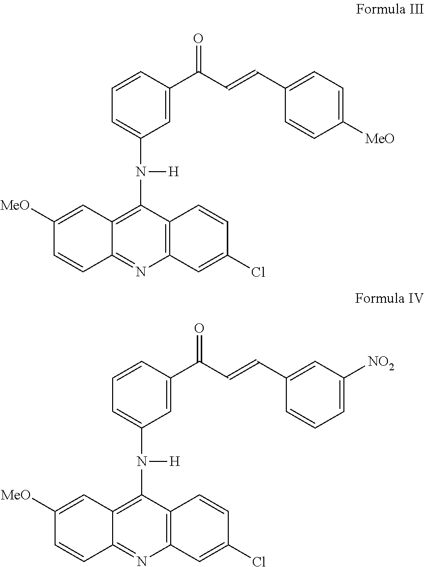 [SMILES: COc1ccc(/C=C/C(=O)c2cccc(Nc3c4ccc(Cl)cc4nc4ccc(OC)cc34)c2)cc1 ; COc1ccc2nc3cc(Cl)ccc3c(Nc3cccc(C(=O)/C=C/c4cccc([N+](=O)[O-])c4)c3)c2c1]

An embodiment of the present invention relates to a process for preparing compound of Formula I comprising reacting a compound of Formula V with a heterocyclic compound preferably having N as a heteroatom to form a mixture and refluxing the mixture in the presence of a solvent to obtain compound of Formula I.

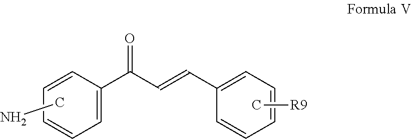

wherein, R9 is selected from an alkoxy group, NO2,

The compound of Formula V is preferably selected from 1-(3-aminophenyl)-3-(4-methoxyphenyl)prop-2-en-1-one or 1-(3-aminophenyl)-3-(4-nitrophenyl)prop-2-en-1-one. The heterocyclic compound is preferably 6,9-dichloro-2-methoxyacridine and the solvent is preferably HCl in ethanol.

An embodiment of the present invention discloses a process to prepare compound of Formula III. The process comprises the steps of reacting (2E)-1-(3-aminophenyl)-3-(4-methoxyphenyl)prop-2-en-1-one with 6,9-dichloro-2-methoxyacridine. The mixture is refluxed in the presence of a solvent, preferably HCl and ethanol to obtain compound of Formula III that is 1-(3-(6-chloro-2-methoxyacridin-9-ylamino)phenyl)-3-(4-methoxyphenyl)prop-2-en-1-one. The reaction mixture is refluxed at a temperature in the range of 40° C. to 100° C. for 2-20 hours to obtain compound of Formula III.

Another embodiment of the present invention discloses a process to prepare compound of Formula IV. The process comprises the steps of reacting (2E)-1-(3-aminophenyl)-3-(4-nitrophenyl)prop-2-en-1-one with 6,9-dichloro-2-methoxyacridine. The mixture is refluxed in the presence of a solvent, preferably HCl in ethanol to obtain compound of Formula IV that is 1-(3-(6-chloro-2-methoxyacridin-9-ylamino)phenyl)-3-(4-nitrophenyl)prop-2-en-1-one. The reaction mixture is refluxed at a temperature in the range of 40° C. to 100° C. for 2-20 hours to obtain compound of Formula I

The compounds as disclosed include the salts, derivatives and other forms thereof.

The compounds of the present invention inhibit proliferation or eradicate cancer cells and/or cancer cells having significant renewal potential, such as cancer stem cells.

In an embodiment of the present invention, a pharmaceutical composition comprises the aforesaid compounds along with suitable pharmaceutical excepients. The excepients are known in the industry and can be selected from sweeteners, flavoring agents, coloring agents, aroma inducing agents, etc.

According to an embodiment of the present invention, a pharmaceutical composition comprises the aforesaid compounds with at least one chemotherapeutic agent such as but are not limited to imatinib, nilotinib, gefitinib, sunitinib, carfilzomib, salinosporamide A, retinoic acid, cisplatin, carboplatin, oxaliplatin, mechlorethamine, cyclophosphamide, chlorambucil, ifosfamide, azathioprine, mercaptopurine, doxifluridine, fluorouracil, gemcitabine, methotrexate, tioguanine, vincristine, vinblastine, vinorelbine, vindesine, podophyllotoxin, etoposide, teniposide, tafluposide, paclitaxel, docetaxel, irinotecan, topotecan, amsacrine, actinomycin, doxorubicin, daunorubicin, valrubicin, idarubicin, epirubicin, plicamycin, mitomycin, mitoxantrone, melphalan, busulfan, capecitabine, pemetrexed, epothilones, 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, Abraxane, Accutane, Actinomycin-D, Adriamycin, Adrucil, Afinitor, Agrylin, Ala-Cort, Aldesleukin, Alemtuzumab, ALIMTA, Alitretinoin, Alkaban-AQ, Alkeran, All-transretinoic Acid, Alpha Interferon, Altretamine, Amethopterin, Amifostine, Aminoglutethimide, Anagrelide, Anandron, Anastrozole, Arabinosylcytosine, Ara-C, Aranesp, Aredia, Arimidex, Aromasin, Arranon, Arsenic Trioxide, Arzerra, Asparaginase, ATRA, Avastin, Azacitidine, BCG, BCNU, Bendamustine, Bevacizumab, Bexarotene, BEXXAR, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine Wafer, Casodex, CC-5013, CCI-779, CCNU, CDDP, CeeNU, Cerubidine, Cetuximab, Chlorambucil, Citrovorum Factor, Cladribine, Cortisone, Cosmegen, CPT-11, Cytadren, Cytosar-U, Cytoxan, Dacarbazine, Dacogen, Dactinomycin, Darbepoetin Alfa, Dasatinib, Daunomycin, Daunorubicin Hydrochloride, Daunorubicin Liposomal, DaunoXome, Decadron, Decitabine, Delta-Cortef, Deltasone, Denileukin, Diftitox, DepoCyt, Dexamethasone, Dexamethasone Acetate, Dexamethasone Sodium Phosphate, Dexasone, Dexrazoxane, DHAD, DIC, Diodex, Docetaxel, Doxil, Doxorubicin, Doxorubicin Liposomal, Droxia, DTIC, DTIC-Dome, Duralone, Efudex, Eligard, Ellence, Eloxatin, Elspar, Emcyt, Epirubicin, Epoetin Alfa, Erbitux, Erlotinib, Erwinia L-asparaginase, Estramustine, Ethyol, Etopophos, Etoposide, Etoposide Phosphate, Eulexin, Everolimus, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab, ozogamicin, Gemzar Gleevec, Gliadel Wafer, GM-CSF, Goserelin, Granulocyte-Colony Stimulating Factor, Granulocyte Macrophage Colony Stimulating Factor, Halotestin, Herceptin, Hexadrol, Hexalen, Hexamethylmelamine, HMM, Hycamtin, Hydrea, Hydrocort Acetate, Hydrocortisone, Hydrocortisone Sodium Phosphate, Hydrocortisone Sodium Succinate, Hydrocortone Phosphate, Hydroxyurea, Ibritumomab, Ibritumomab, Tiuxetan, Idamycin, Idarubicin Ifex, IFN-alpha, Ifosfamide, IL-11, IL-2, Imatinib mesylate, Imidazole Carboxamide, Interferon alfa, Interferon Alfa-2b (PEG Conjugate), Interleukin-2, Interleukin-11, Intron A (interferon alfa-2b), Iressa, Irinotecan, Isotretinoin, Ixabepilone, Ixempra, Kidrolase, Lanacort, Lapatinib, L-asparaginase, LCR, Lenalidomide, Letrozole, Leucovorin, Leukeran, Leukine, Leuprolide, Leurocristine, Leustatin, Liposomal Ara-C, Liquid Pred, Lomustine, L-PAM, L-Sarcolysin, Lupron, Lupron Depot, Matulane, Maxidex, Mechlorethamine, Mechlorethamine Hydrochloride, Medralone, Medrol, Megace, Megestrol, Megestrol Acetate, Melphalan, Mercaptopurine, Mesna, Mesnex, Methotrexate, Methotrexate Sodium, Methylprednisolone, Meticorten, Mitomycin, Mitomycin-C, Mitoxantrone, M-Prednisol, MTC, MTX, Mustargen, Mustine, Mutamycin, Myleran, Mylocel, Mylotarg, Navelbine, Nelarabine, Neosar, Neulasta, Neumega, Neupogen, Nexavar, Nilandron, Nilotinib, Nilutamide, Nipent, Nitrogen Mustard, Novaldex Novantrone, Nplate, Octreotide, Octreotide acetate, Ofatumumab, Oncospar, Oncovin, Ontak, Onxal, Oprelvekin, Orapred, Orasone, Oxaliplatin, Paclitaxel, Paclitaxel Protein-bound, Pamidronate, Panitumumab, Panretin, Paraplatin, Pazopanib, Pediapred, PEG Interferon, Pegaspargase, Pegfilgrastim, PEG-INTRON, PEG-L-asparaginase, PEMETREXED, Pentostatin, Phenylalanine Mustard, Platinol, Platinol-AQ, Prednisolone, Prednisone, Prelone, Procarbazine, PROCRIT, Proleukin, Prolifeprospan 20 with Carmustine Implant, Purinethol, Raloxifene, Revlimid, Rheumatrex, Rituxan, Rituximab, Roferon-A (Interferon Alfa-2a), Romiplostim, Rubex, Rubidomycin hydrochloride, Sandostatin, Sandostatin LAR, Sargramostim, Solu-Cortef, Solu-Medrol, Sorafenib, SPRYCEL, STI-571, Streptozocin, SU11248, Sunitinib, Sutent, Tamoxifen, Tarceva, Targretin, Tasigna, Taxol, Taxotere, Temodar, Temozolomide, Temsirolimus, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Torisel, Tositumomab, Trastuzumab, Treanda, Tretinoin, Trexall, Trisenox, TSPA, TYKERB, VCR, Vectibix, Velban, Velcade, VePesid, Vesanoid, Viadur, Vidaza, Vinblastine, Vinblastine Sulfate, Vincasar Pfs, Vincristine, Vinorelbine, Vinorelbine tartrate, VLB, VM-26, Vorinostat, Votrient, VP-16, Vumon, Xeloda, Zanosar, Zevalin, Zinecard, Zoladex, Zoledronic acid, Zolinza, Zometa, or combinations thereof.

The compounds can be administered orally, parenterally, by inhalation spray, topically, rectally, nasally, buccally, vaginally or via an implanted reservoir. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. In addition, sterile, fixed oils are conventionally employed as a solvent or suspending medium.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

An embodiment of the present invention discloses a method of inhibition of unregulated cell growth such as cancer cells and/or cancer cells having significant renewal potential, such as cancer stem cells in a patient by administering the compounds or salts thereof or the compositions in an effective amount.

Another embodiment of the invention discloses the use of the compounds in the inhibition or eradication of unregulated cell growth such as cancer cells and/or cancer cells having significant renewal potential, such as cancer stem cells.

The compounds can be used for the treatment of breast cancer, prostate cancer, brain cancer, blood cancer, bone marrow cancer, liver cancer, pancreas cancer, skin cancer, kidney cancer, colon cancer, ovary cancer, lung cancer, testicle cancer, penis cancer, thyroid cancer, parathyroid cancer, pituitary cancer, thymus cancer, retina cancer, uvea cancer, conjunctiva cancer, spleen cancer, head cancer, neck cancer, trachea cancer, gall bladder cancer, rectum cancer, salivary gland cancer, adrenal gland cancer, throat cancer, esophagus cancer, lymph nodes cancer, sweat glands cancer, sebaceous glands cancer, muscle cancer, heart cancer, and stomach cancer.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### EXAMPLES

The examples illustrated herein below define the invention but are not limiting thereof:

### Example 1

### 1-(3-(6-chloro-2-methoxyacridin-9-ylamino)phenyl)-3-(4-methoxyphenyl)prop-2-en-1-one

0.487 gm of (2E)-1-(3-aminophenyl)-3-(4-methoxyphenyl)prop-2-en-1-one with 1 gm of 6,9-dichloro-2-methoxyacridine. 0.1 ml of HCl and 50 ml of ethanol was added to the above mixture The mixture was refluxed at 80° C. for 14 hours. The reaction mixture was concentrated on roteva evaporator under vacuum to obtain a dark yellow coloured solid of crude 1-(3-(6-chloro-2-methoxyacridin-9-ylamino)phenyl)-3-(4-methoxyphenyl)prop-2-en-1-one. The dark yellow solid was charged with 70 ml methanol, 5.40 gm of para-methoxy benzaldehyde and 0.144 gm of NaOH for 14 hours to obtain a precipitate. The precipitate was washed with 30 ml methanol and dried at 100° C. for 3 hours. The dried product was recrystallized with ethanol to obtain an orange red colored solid of pure 1-(3-(6-chloro-2-methoxyacridin-9-ylamino)phenyl)-3-(4-methoxyphenyl)prop-2-en-1-one.

### Example 2

### 1-(3-(6-chloro-2-methoxyacridin-9-ylamino)phenyl)-3-(4-nitrophenyl)prop-2-en-1-one.

0.487 gm of (2E)-1-(3-aminophenyl)-3-(4-nitrophenyl)prop-2-en-1-one with 1 gm of 6,9-dichloro-2-methoxyacridine. 0.1 ml of HCl and 50 ml of ethanol was added to the above mixture The mixture was refluxed at 80° C. for 14 hours. The reaction mixture was concentrated on roteva evaporator under vacuum to obtain a dark yellow coloured solid of crude 1-(3-(6-chloro-2-methoxyacridin-9-ylamino)phenyl)-3-(4-nitrophenyl)prop-2-en-1-one. The dark yellow solid was charged with 70 ml methanol, 1.09 gm of para-nitro benzaldehyde and 0.144 gm of NaOH for 14 hours to obtain a precipitate. The precipitate was washed with 30 ml methanol and dried at 100° C. for 3 hours. The dried product was recrystallized with ethanol to obtain an orange red colored solid of pure 1-(3-(6-chloro-2-methoxyacridin-9-ylamino)phenyl)-3-(4-nitrophenyl)prop-2-en-1-one.

### In Vitro Colorimetric Cell Death Assay (MTT Assay)

MTT assay was carried for assessing cell viability where the cells were grown in two-dimensional surface.

The procedure for the assay is as follows. Cancer cells were plated in 96 well plates as per predetermined plating efficiency. The plates were incubated for 24 hours in a 5% CO2 atmosphere at 37° C., a range of concentrations of the compound of the present invention was added to the wells, the plates were incubated further for 48 hours in a 5% CO2 atmosphere, the plates were centrifuged twice at 3000 rpm for 3 minutes, the supernatant fluid was discarded, 100 uL of 0.5 mg/mL MIT solution was added and the plates were incubated for 4 hours in a 5% CO2 atmosphere at 37° C. The plates were then centrifuged twice at 3000 rpm for 3 minutes, supernatant was aspirated very carefully, 200 uL DMSO was added to each well to solubilize MTT crystals and mixed well by shaking the plates, the plates were incubated for 10 minutes in a 5% CO2 atmosphere at 37° C., the plates were placed on the shaker of an ELISA plate reader and the absorbance at 570 nm was measured, then the percentage of viable cells remaining was calculated by first subtracting the background absorbance then comparing to the absorbance of a non-drug-treated cell sample, and the results were plotted on a graph to determine the IC50 for the compound as known in the art. The reference drug used was Cisplatin.

The results of the in vitro colorimetric cell death assay are given in Table 1, Table 2, Table 3, Table 4, Table 5, Table 6 and Table 7.

A. Colon Cancer

TABLE 1

| | |
|---|---|
| | Compound of |
| Cisplatin | Formula IV |

| | |
|---|---|
| Cell Line | IC50 (uM) |

| | | |
|---|---|---|
| HCT-15 | 49.14 | 2.405 |
| Colo205 | 44.34 | 8.4 |
| Colo320 | 38.34 | 2.5 |

B. Cervical Cancer

TABLE 2

| | |
|---|---|
| | Compound of |
| Cisplatin | Formula IV |

| | |
|---|---|
| Cell Line | IC50 (uM) |

| | | |
|---|---|---|
| SiHa | 43.21 | 3.78 |

C. Lung Cancer

TABLE 3

| | |
|---|---|
| | Compound of |
| Cisplatin | Formula IV |

| | |
|---|---|
| Cell Line | IC50 (uM) |

| | | |
|---|---|---|
| NCIH23 | 45.72 | 2.52 |

D. Fibroblast

TABLE 4

| | |
|---|---|
| | Compound of |
| Cisplatin | Formula IV |

| | |
|---|---|
| Cell Line | IC50 (uM) |

| | | |
|---|---|---|
| L929 | 38.02 | 1.94 |

E. Hepatic Cancer

TABLE 5

| | |
|---|---|
| | Compound of |
| Cisplatin | Formula IV |

| | |
|---|---|
| Cell Line | IC50 (uM) |

| | | |
|---|---|---|
| Hep3B | 37.58 | 2.11 |

F. Breast Cancer

TABLE 6

| | |
|---|---|
| | Compound of |
| Cisplatin | Formula IV |

| | |
|---|---|
| Cell Line | IC50 (uM) |

| | | |
|---|---|---|
| T47D | 95.5 | 28.1 |
| MDA MB 231 | 75.16 | 3.14 |

G. Prostate

Cancer

TABLE 7

| | |
|---|---|
| | Compound of |
| Cisplatin | Formula IV |

| | |
|---|---|
| Cell Line | IC50 (uM) |

| | | |
|---|---|---|
| LnCaP | 38.39 | 3.08 |

As can be seen from Table 1, Table 2, Table 3, Table 4, Table 5, Table 6 and Table 7, the compound of Formula IV is much more effective in the inhibition of cancer cells than the reference compound Cisplatin. The values of IC50 for Formula IV are much less than Cisplatin indicating that the compound is effective in the treatment of various types of Cancer.

In Vitro 3D Sphere Forming Stem Cell Assay Compounds of Formula III & IV were evaluated in an in vitro 3D sphere forming stem cell assay as described by Rajasekhar, V K, Analytical Methods for Cancer Stem Cells. Methods in Molecular Biology, 2007, Volume 407, 83-95.

An exemplary procedure for the in vitro 3D sphere forming stem cell assay is described as follows. Breast Cancer Cells were grown in two dimensions on a plastic substrate, harvested in suspension in serum-free media, then the cells in the sample were trypsinized and a single cell suspension was formed by passing through a cell strainer. The cells were diluted according to the predetermined plating efficiency for the cell line being studied by suspending the cells in stem cell culture medium. 100 uL of this suspension was added into each well of a 96 well suspension plate, and the plate was incubated at 37° C. in 5% CO₂ atmosphere for 24 hours, then 2 uL of appropriate concentrations of the drugs was added into each respective well along with 100 uL of stem cell culture medium, and the plates incubated at 37° C. under 5% CO₂ atmosphere for 72 hours. 2.5 uL of the appropriate drug concentration (Formula III and Formula IV) of was then added to each respective well along with 50 uL of stem cell culture medium, then the plates incubated at 37° C. under 5% CO₂ atmosphere for 72 hours. 3 uL of the appropriate drug concentration was added to each respective well along with 50 uL of stem cell culture medium, then incubated at 37° C. under 5% CO₂ atmosphere for 72 hours, the spheres formed were observed under a microscope then counted and scored by size.

Results of the in vitro 3D sphere forming stem cell assay are set forth in Table 8. The number in each box is the total number of spheres formed in the presence of either cisplatin, compound of Formula III or compound of Formula IV at each drug concentration. GC refers to a growth control performed in the absence of drug or solvent (DMSO). GCD refers to a growth control performed in the absence of drug, but in the presence of DMSO.

TABLE 8

| | Dilution | | | | | |
|---|---|---|---|---|---|---|
| Cell | (from stock | # of 3D Spheres Formed (±SD) | | | | |
| Line | of 0.044M) | 110 uM | 11 uM | 1.1 uM | GC | GCD |
| MDAMB | Cisplatin | 16(±2) | 24(±2) | 35(±4) | 17(±3) | 27(±2) |
| 231 | Formula IV | 0(±0) | 0(±0) | 9(±1) | 23(±3) | 19(±2) |
| | Formula III | 0(±0) | 0(±0) | 15(±2) | 17(±3) | 27(±2) |

Significant sphere reduction was observed for compounds of Formula III and Formula IV as compared to standard drug Cisplatin and therefore compounds of Formula III and Formual IV are more potent compounds compared to Cisplatin.

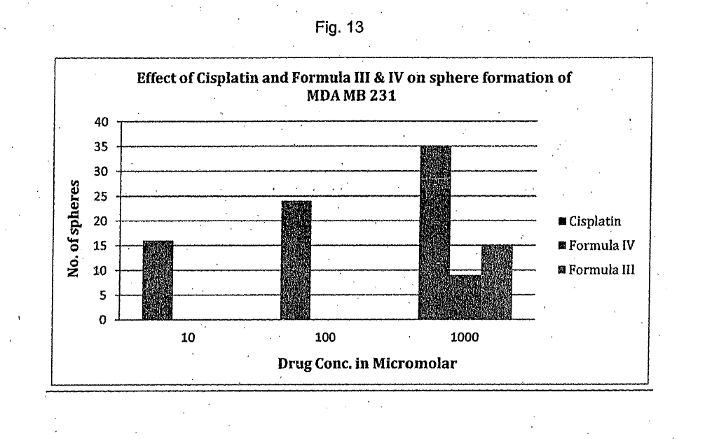

FIG. 13 shows the effect of Cisplatin and Formula III & IV on sphere formation of MDA MB 231 as presented in the above Table.

### In Vitro Soft Agar Colony Forming Growth Assay

Compounds of Formula IV were evaluated in an in vitro soft agar colony forming growth inhibition assay as described by Lieber, M M, and Kovach, J S. Soft agar colony formation assay for chemotherapeutic sensitivity of human solid tumors. Mayo Clin Proc 1982; 57: 527-528 and Tanigawa, N, Kern, D H, Kikasa, Y, and Morton, D L. Rapid assay for evaluating the chemosensitivity of human tumors in soft agar culture. Cancer Res 1982; 42: 2159-2164.

An exemplary procedure for the in vitro soft agar colony forming growth inhibition assay is as follows. A mixture of 50 uL of 2× medium and 50 uL of 1.2% Bacto Agar was plated onto each well of a 96 well microtiter assay plate, 10 uL of cells (of specific plating efficiency pre-standardized per cell line) were mixed with 20 uL of 2× medium and 30 uL of 0.8% Bacto Agar and 1.6 uL of compound of Formula IV in a vial, the drug/cell mixture was transferred to the solidified agar layer of each respective well of the plate, the plate was incubated at 37° C. in 5% CO₂ for one week (feeding each well after 3 days with 50 uL of 2× medium), then 16 uL of Alamar Blue (1.5 mg/mL) was added to each well, the absorbance of each well was measured at 630 nm and percent viability of each well relative to the absorbance reading of the growth control well without drug was calculated, and the IC50 of the compound was determined.

Results of the in vitro soft agar assay are set forth in Table 9.

TABLE 9

| | Cisplatin | Formula IV |
|---|---|---|
| Cell Line | IC50 (uM) | IC50 (uM) |
| MCF 7 | 38.9 | 1.47 |
| MDA MB 231 | 38.9 | 2.52 |
| T47D | 39.81 | 3.3 |
| DU145 | 33.11 | 2.86 |
| PC3 | 52.48 | 2.46 |
| LNCAP | 31.62 | 2.17 |
| HeLa | 3.5 | 2.4 |
| SiHa | 19.495 | 2.51 |
| L929 | 9.44 | 0.16767 |
| HCT-15 | 39.96 | 2.58 |

The compound of Formula IV shows potent anticancer activity in Breast Cancer Cell lines (MCF 7, MDA MB 231, T47D), Prostate cancer cell (PC3, DU145 & LNCaP), Cervical cancer cell (HeLa& SiHa), Fibroblast cell line (L929) and Colon cancer cell line (HCT-15). The compound of Formula IV is much more effective in the treatment of cancer than the standard therapeutic drug Cisplatin. The lower IC50 values of Formula IV indicate that the compound of Formula IV is highly potent in treating various types of cancer.

A study was conducted to determine the effect of compound of Formula IV and Cisplatin on CD44 and CD24 expression on PC3 (Prostrate Cancer) cells by Flow Cytometry.

Procedure:

PC3 cells (0.35×106) were cultured in R.P.M.I (Roswell Park Memorial Institute Medium)-1640 cell culture medium with 10% F.B.S (Fetal Bovine Serum) on 60 mm TC (Tissue Culture) plates. Cells were exposed to IC10 drug concentration of Cisplatin & IC10 drug concentration of compound of Formula IV in duplicates. Similarly other sets were exposed to IC25 drug concentration of Cisplatin & compound of Formula IV in duplicates. IC10 and IC25 drug concentrations were calculated as per MTT results. All the sets were incubated at 37° C., 5% CO2 for 48 hours. After 48 hours, cells were observed under the microscope. Cells were trypsinised, washed with D.P.B.S. (Dulbecco's Phosphate Buffered Saline) and 50 μL of cells were taken for each set and 5 μL of each of CD24 and CD44 antibodies were added. CD24-FITC (Fluorescein isothiocyanate) labeled & CD44-PE (Phycoerythrin) labeled was used for the study. The sets were incubated for 45 minutes at 4° C. in dark for proper binding of antibodies. After incubation the cells were washed with 200 μL of D.P.B.S., supernatant was discarded and the cells were finally suspended in 300 μL of FACS buffer (4% F.B.S in D.P.B.S.). Samples were kept at 4° C. in dark till they were acquired on FACS (Fluorescent-Activated Cell Sorting). Acquisition was done on BD-FACS Accuri C6.

Results:

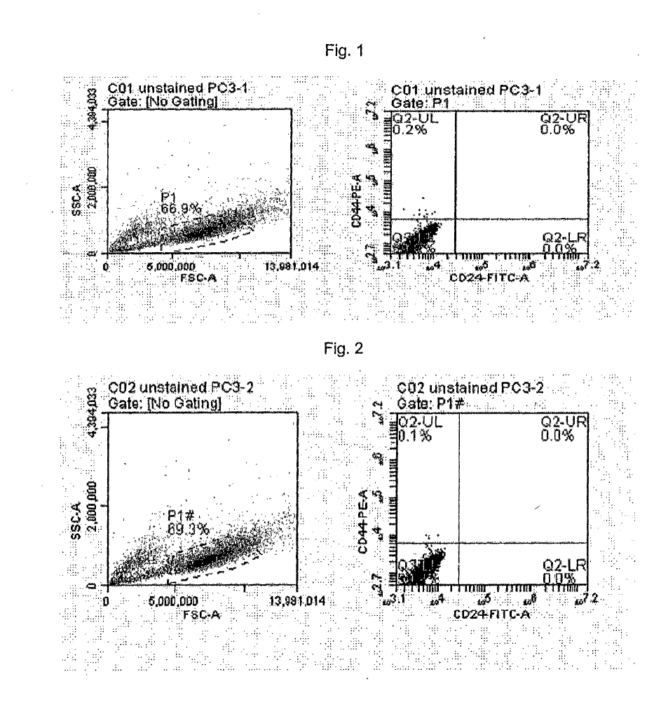

The unstained sample (without compound of Formula IV) of PC3 was run to gate the live cell, population. The sample gate was used throughout acquisition of all samples. A quadrant plot was made to distinguish between various cell populations. Cells in the Lower Left (LL) of the plot as indicated in FIGS. 1-12 represent negative population (population negative for CD44 and CD24). Cells in the Lower Right (LR) plot represent cell population expressing CD24 population. Cells in the Upper Left (UL) region express CD44 population and the cells in the Upper Right (UR) express cell population positive for both CD44 and CD24.

CD44 is highly expressed on cancer stem cells, it's eradication is an indication of inhibition of cancer stem cells and consequently the effect of the compounds on the eradication of cancer stem cells.

FIG. 1 and FIG. 2 represent unstained PC3 population. As expressed in SSC-A & FSC-A plot only live cell population (66.9% & 69.3% have been gated in the P1 region) and the debri has been excluded. The gated population is represented as negative population in the quadrant plot.

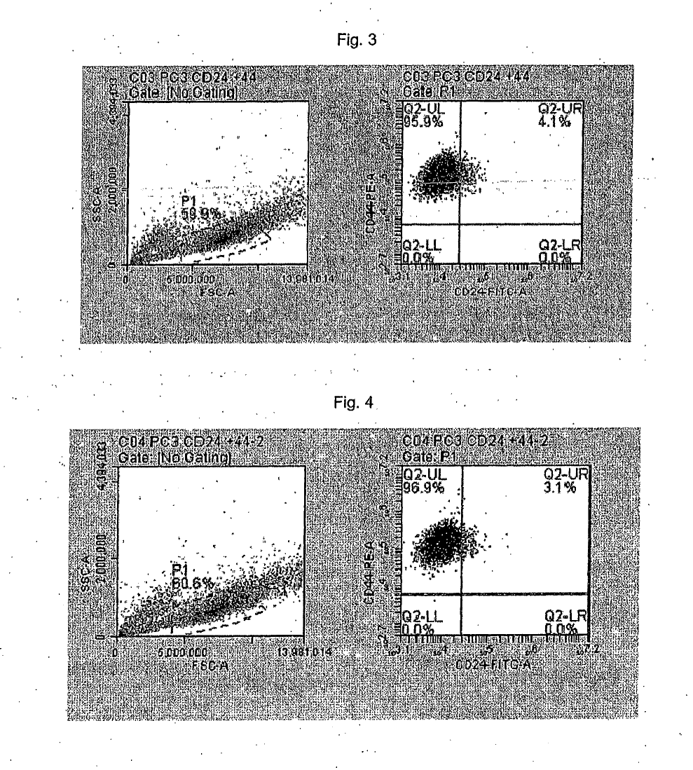

FIG. 3 and FIG. 4 represent PC3 cells with CD24 & CD44 antibodies added. As can be seen in the quadrant plot, most of the PC3 cells (95.9% & 96.9%) represent CD44 expression and 4.1% & 3.1% cells show co-expression for CD44 and CD24.

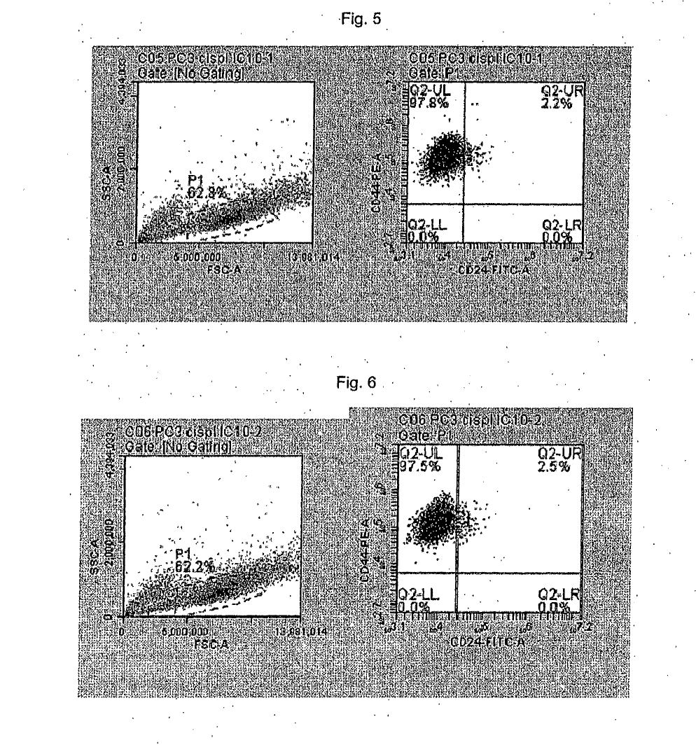

FIG. 5 and FIG. 6 represent PC3 cells exposed to IC10 Cisplatin. As can be seen, most of the cells remain unaffected even after the treatment.

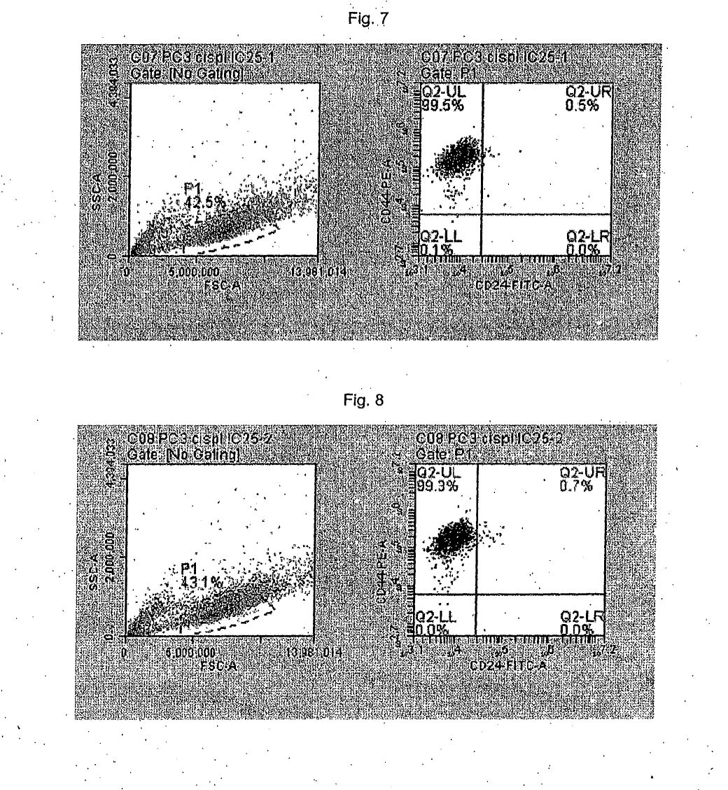

FIG. 7 and FIG. 8 represent PC3 cells exposed to IC25 drug concentration of Cisplatin. As can be seen, there is a good number of CD44 cell population surviving even after the treatment.

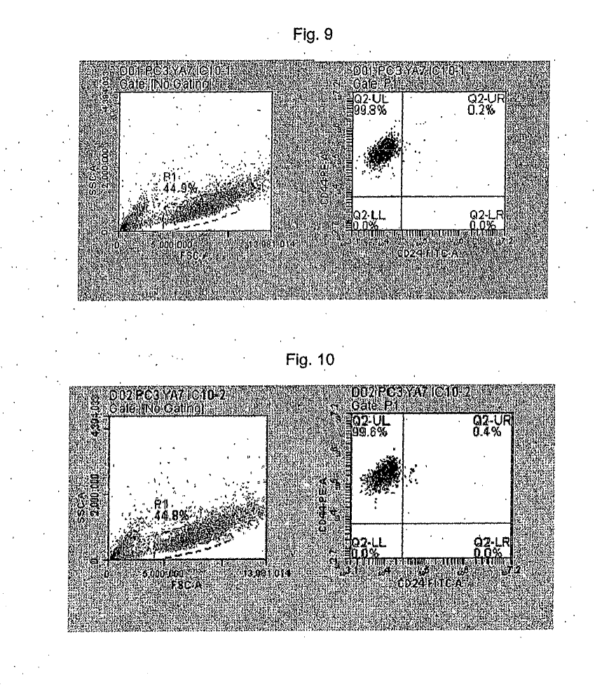

FIG. 9 and FIG. 10 represent PC3 cells exposed to IC10 drug concentration of YA7. As can be seen, there is a reduction in viable cell population (44.9% & 44.8% respectively). There is still a good number of CD44+ cell population.

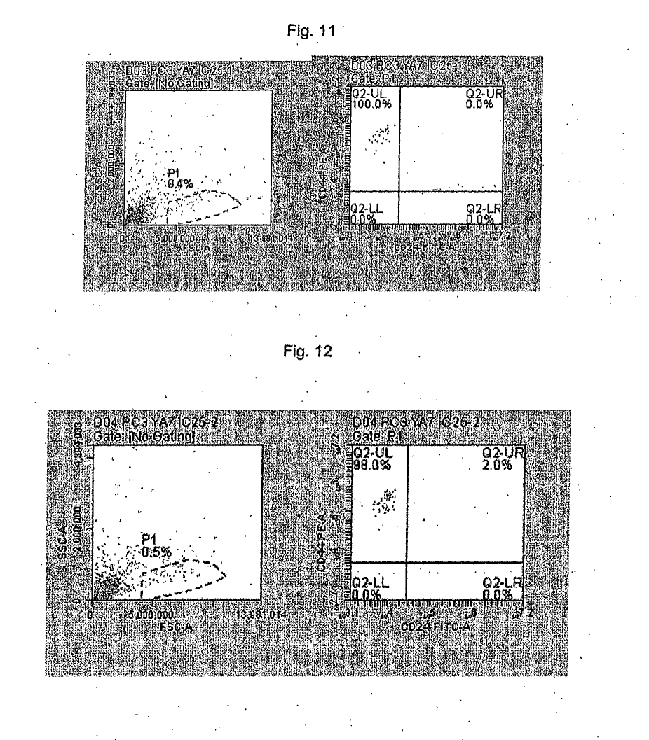

FIG. 11 and FIG. 12 represent PC3 cells exposed to IC25 drug concentration of compound of Formula IV. As can be seen, there are very few cells surviving after IC25 treatment (0.4% and 0.5% respectively). Most of the CD44 population has been killed, thus indicating that compound of Formula IV is effective in the treatment of prostate cancer.

The compounds of the present invention have wide applicability in the treatment of various conditions. The compounds or their compositions have broad-spectrum applications in the treatment of cancer of breast, prostrate, cervical, brain, blood, bone marrow, liver, pancreas, skin, kidney, colon, ovary, lung, testicle, penis, thyroid, parathyroid, pituitary, thymus, retina, uvea, conjunctiva, spleen, head, neck, trachea, gall bladder, rectum, salivary gland, adrenal gland, throat, esophagus, lymph nodes, sweat glands, sebaceous glands, muscle, heart, and stomach. The compounds or their compositions may also be used to treat other conditions such as cerebral disorders, cardiovascular disease and related disease states, including cholesterol or lipid related disorders, such as, e.g., atherosclerosis an autoimmune disorder, a neurodegenerative or neurological disorder, schizophrenia, a bone-related disorder, liver disease, or a cardiac disorder. The exact amount required will vary from subject to subject, depending on the species, age, and general condition of the subject, the severity of the infection, the particular agent, its mode of administration, and the like.

These compounds exhibit no activity on normal cells but nearly 100 fold times enhanced activity on cancer cells and cancer stem cells.

1. Compound of Formula I 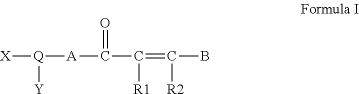
wherein,
X is substituted or unsubstituted heterocyclic ring, substituted or unsubstituted tricyclic ring with a heteroatom, preferably N;
Q is preferably N;
Y is H;
A is substituted or unsubstituted aromatic ring, substituted or unsubstituted aromatic ring with a heteroatom;
R1 and R2, each independently is H;
B is a substituted or unsubstituted aromatic ring and the salts thereof.

2. The compound as claimed in claim 1, wherein the compound is: 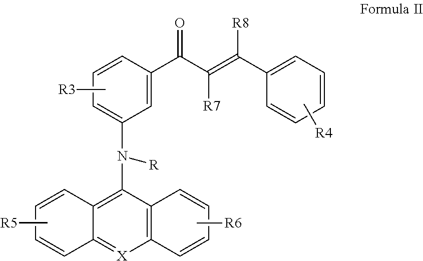
wherein,
R3 is H, I, Br, alkoxy group, optionally substituted alkyl group;
R4, R5 and R6, each independently is alkoxy, I, Cl, Br, CN, NO2, optionally substituted alkyl group;
R7 and R8 each independently is H
X is N
R is H, HCl, H₂SO₄;
or salts thereof.

3. The compound as claimed in claim 2, wherein the compound is:

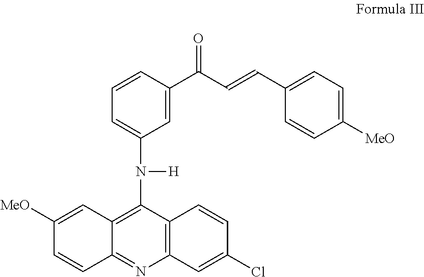 [SMILES: COc1ccc(/C=C/C(=O)c2cccc(Nc3c4ccc(Cl)cc4nc4ccc(OC)cc34)c2)cc1]

4. The compound as claimed in claim 2, wherein the compound is:

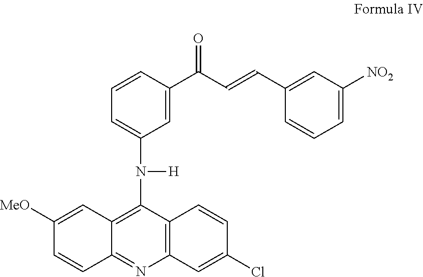 [SMILES: COc1ccc2nc3cc(Cl)ccc3c(Nc3cccc(C(=O)/C=C/c4cccc([N+](=O)[O-])c4)c3)c2c1]

5. A pharmaceutical composition comprising the compound or a salt of the compound as claimed in claim 1 or salts thereof with pharmaceutically acceptable excepients.

6. A pharmaceutical composition comprising the compound or a salt of the compound as claimed in claim 1 or salts thereof with at least one chemotherapeutic agent.

7. A process for preparing a compound for inhibition of unregulated cell growth, the process comprising
reacting a compound of Formula V with a heterocyclic compound preferably having N as a heteroatom to form a mixture; and
refluxing the mixture in the presence of a solvent to obtain the compound.

8. The process as claimed in claim 7, wherein the compound of Formula V is selected from 1-(3-aminophenyl)-3-(4-methoxyphenyl)prop-2-en-1-one or 1-(3-aminophenyl)-3-(4-nitrophenyl)prop-2-en-1-one.

9. The process as claimed in claim 7, wherein the heterocyclic compound is preferably 6,9-dichloro-2-methoxyacridine.

10. The process as claimed in claim 7, wherein the solvent is preferably HCl in ethanol.

11. A method of inhibiting proliferation or eradication of cancer cells and/or cancer cells having significant renewal potential, such as cancer stem cells comprising administering the compound as claimed in claim 1 or a salt thereof to a patient in need thereof.

12. A method of inhibiting proliferation of or eradication of cancer cells and/or cancer cells having significant renewal potential, such as cancer stem cells in a patient in need thereof comprising administering to said patient a composition as claimed in claim 5.

13. A method of inhibiting proliferation of or eradication of cancer cells and/or cancer cells having significant renewal potential, such as cancer stem cells in a patient in need thereof comprising administering to said patient a composition as claimed in claim 6.
